(19) United States
(12) Patent Application Publication
(10) Pub. No.: US 2020/0261723 A1
(43) Pub. Date: Aug. 20, 2020

(54) IMPLANT PACKAGE FOR COCHLEAR PROSTHESIS AND METHOD OF IMPLANTING THE SAME

(71) Applicant: TODOC CO., LTD., Seoul (KR)

(72) Inventors: Seung Ha OH, Seoul (KR); Kyou-Sik MIN, Suwon-si (KR); Doo Hee KIM, Seoul (KR)

(21) Appl. No.: 16/279,440

(22) Filed: Feb. 19, 2019

(51) Int. Cl. A61N 1/36; A61N 1/05

(52) U.S. Cl. CPC A61N 1/36038; A61N 1/0541

(57) ABSTRACT

An implant package for a cochlear prosthesis is implanted in a head of a person to be adjacent to an extracorporeal device. An inner coil unit receives an electrical signal generated by an audio processor. A main body is connected to the inner coil unit to convert the electrical signal into an electrical stimulation signal. An electrode unit is connected to the main body and inserted into a cochlea to stimulate auditory nerves of the cochlea using the electrical stimulation signal. The inner coil unit may be separated from the main body to be disposed between skin and muscle of the head. The coil within the head is separated from the main body to be disposed between the skin and the muscle to improve communications efficiency for electrical signals.

### BACKGROUND OF THE INVENTION

### Field of the Invention

The present disclosure relates generally to an implant package for a cochlear prosthesis and, more particularly, to an implant package for a cochlear prosthesis and a method of implanting the same, in which a coil can be freely disposed.

### Description of the Related Art

The information disclosed in the Background of the Invention section is only for the enhancement of understanding of the background of the invention, and should not be taken as an acknowledgment or as any form of suggestion that this information forms a prior art that would already be known to those skilled in the art.

A cochlear prosthesis is a device implanted in a cochlea of a hearing impaired person. When the hearing of the hearing impaired person having a disease in the cochlea is not improved by a hearing aid, the cochlear prosthesis is implanted in the cochlea of the hearing impaired person to stimulate the cochlea by converting external sound into stimulation signals and stimulate the cochlea using the stimulation signals.

However, cochlear prostheses of the related art have the following drawbacks. Electric signals may not be properly transferred due to a long distance between an outer coil outside of the temporal region and an inner coil within a muscular tissue. A wide area of bone tissue must be removed so that a main body of a cochlear prosthesis can fitted to the bone tissue. The position of the inner coil is fixed by the position of the fitted main body, so that the position of the outer coil aligned with the inner coil is also limited.

In this regard, Korean Utility Model Application Publication No. 20-2010-0006338 discloses an audio processor for a cochlear prosthesis and a cochlear prosthesis including the same, while Korean Registered Utility Model No. 20-0449559 discloses an audio processor for a cochlear prosthesis and a cochlear prosthesis including the same.

### RELATED ART DOCUMENT

Patent Document 1: Korean Utility Model Application Publication No. 20-2010-0006338
Patent Document 2: Korean Registered Utility Model No. 20-0449559

### SUMMARY OF THE INVENTION

Accordingly, the present disclosure has been made keeping in mind the above problems occurring in the related art, and the present disclosure proposes an implant package for a cochlear prosthesis, in which a coil within the head is separated from a main body to be disposed between skin and muscle to improve communications efficiency for electrical signals.

Also provided is a method of implanting the implant package for a cochlear prosthesis in a body portion of a person.

The object of the present disclosure is not limited to the aforementioned description, and other objects not explicitly disclosed herein will be clearly understood by those skilled in the art to which the present disclosure pertains from the description provided hereinafter.

In order to achieve the above object, according to one aspect of the present disclosure, there is provided an implant package for a cochlear prosthesis. The implant package may be implanted in a head of a person to be adjacent to an extracorporeal device including an audio processor converting sound into an electrical signal and an outer coil unit transmitting the electrical signal into the head. The implant package may include: an inner coil unit receiving the electrical signal transmitted by the outer coil unit; a main body connected to the inner coil unit to convert the electrical signal into an electrical stimulation signal; and an electrode unit connected to the main body and inserted into a cochlea to stimulate auditory nerves of the cochlea using the electrical stimulation signal. The inner coil unit may be separated from the main body to be disposed between skin and muscle of the head.

The main body may be connected to the inner coil unit via a conductive wire, and may be separated from the inner coil unit to be implanted in a bone tissue of the head between the muscle and the bone tissue of the head.

The inner coil unit may be disposed in a location in which the bone tissue has a flat surface.

The inner coil unit may be disposed in a location adjacent to or spaced apart from the main body.

The inner coil unit may be disposed in the external auditory canal, a location adjacent to the external auditory canal, or the mastoid without skin incision or tissue peeling.

According to another aspect of the present disclosure, a method of implanting a cochlear prosthesis may include: sequentially incising portions of the skin, a muscle, and a bone tissue of a temporal region, followed by mastoid cavity removal and then posterior or round window membrane excision; removing a portion of the bone tissue, in which a main body connected to a reference electrode and a stimulation electrode to convert an electrical sound signal into an electrical stimulation signal is to be located, along the shape of the main body, and fixing the main body to the bone tissue; disposing a wire-shaped connector connected to the main body and an inner coil of an inner coil unit between the skin and the muscle by extending the connector and the inner coil unit through the muscle; and inserting the stimulation electrode generating the stimulation signal into the cochlea, connecting the reference electrode to a predetermined location, covering a portion into which stimulation electrode is inserted with a soft tissue including the muscle, and suturing the incised portions of the temporal region.

The method may further include attaching an outer coil unit attached to an outer portion of the head of a person to be adjacent to the inner coil unit, the outer coil unit being connected to an audio processor for converting sound into electrical signals to transmit the electrical signals into the head.

In addition, in the disposing of the inner coil unit, the inner coil unit may be disposed in an external auditory canal, a location adjacent to the external auditory canal, or a mastoid without skin incision or tissue peeling, instead of being disposed between the skin and the muscle.

Furthermore, in the fixing of the main body, the main body may be disposed in a location of the head of a person, in which the thickness of the muscle is relatively low and the bone tissue is flat.

According to an embodiment of the present disclosure, the implant package for a cochlear prosthesis may be advantageously configured to reduce the distance between an inner coil and an outer coil, such that transfer efficiencies for electrical signals can be improved.

In addition, the implant package for a cochlear prosthesis may be advantageously configured such that the inner coil and a main body for converting electrical signals into electrical stimulation signals and transmitting the electrical stimulation signals to an electrode unit are separated from each other. Accordingly, it is possible to advantageously change the position of the coil depending on the physical characteristic of a person implanted with implant package.

Furthermore, the method of implanting the implant package for a cochlear prosthesis can implant the implant package by minimizing removal of skin and bone tissue, thereby advantageously reducing a time required for implanting the main body to the bone tissue.

Since the effects of the present disclosure are exerted by contents described herein, regardless of perception of the inventors, the above-described effects are merely specific examples of the effects obtainable from the present disclosure and should not be understood as all effects that the inventors have perceived or are obtainable from the present disclosure.

In addition, the effects of the present disclosure should be determined additionally on the basis of the complete description of the specification. Although not explicitly described, any effects that may be recognized by those skilled in the art to which the present disclosure relates, on the basis of the description of the specification, shall be regarded as the effects described herein.

### BRIEF DESCRIPTION OF THE DRAWINGS

The above and other objects, features and advantages of the present disclosure will be more clearly understood from the following detailed description when taken in conjunction with the accompanying drawings, in which:

FIG. 1 is a block diagram illustrating an implant package for a cochlear prosthesis according to an embodiment of the present disclosure;

FIG. 2 is a perspective view illustrating the implant package for a cochlear prosthesis illustrated in FIG. 1;

FIG. 3 is a schematic view illustrating the implant package for a cochlear prosthesis illustrated in FIG. 1, implanted in the body of a person;

FIG. 4 is a perspective view illustrating a process of implanting the implant package for a cochlear prosthesis illustrated in FIG. 2 in the body of a person; and

FIG. 5 is a flowchart illustrating a method of implanting the implant package for a cochlear prosthesis illustrated in FIG. 1.

### DETAILED DESCRIPTION OF THE INVENTION

Hereinafter, an implant package for a cochlear prosthesis according to exemplary embodiments will be described, in relation to configurations, operations, and effects thereof, with reference to the drawings. For reference, in the drawings, components are omitted or schematically illustrated for the sake of convenience and clarity, and the size of each component may not reflect the actual size. Throughout this document, the same reference numerals and symbols will be used to designate the same or like components. In individual drawings, reference numerals of the same components will be omitted.

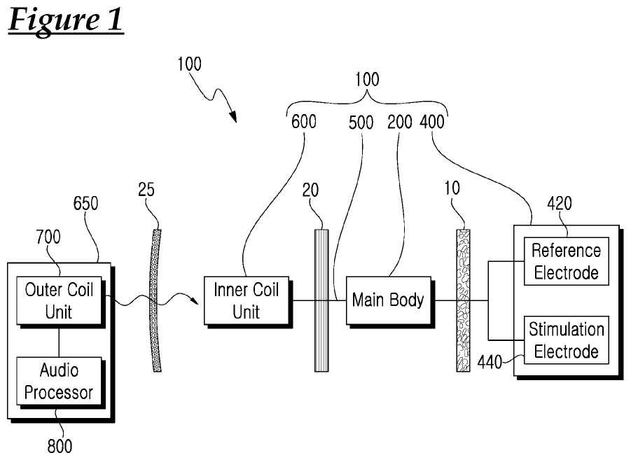

FIG. 1 is a block diagram illustrating an implant package for a cochlear prosthesis according to an embodiment of the present disclosure.

As illustrated in FIG. 1, an implant package for a cochlear prosthesis (hereinafter, referred to as a “cochlear prosthesis implant package”) 100 includes a main body 200, an electrode unit 400, a connector 500, and an inner coil unit 600. An extracorporeal device 650 includes an outer coil unit 700 and an audio processor 800.

The cochlear prosthesis implant package 100 is a component of a cochlear prosthesis, and is wirelessly connected to the extracorporeal device 650 attached to an outer portion of the head of a person. The function of transmitting and receiving electrical signals between the extracorporeal device 650 and the cochlear prosthesis implant package 100 is improved, so that sound can be transferred clearly.

Specifically, in the cochlear prosthesis implant package 100, the main body 200 and the inner coil unit 600 are separated, unlike a structure of the related art in which a main body and an inner coil unit are connected. The main body 200 and the inner coil unit 600 are disposed in a space between a bone tissue 10 and a muscle 20 and a space between the muscle 20 and the skin 25, respectively, such that electrical signals can be effectively transmitted from the outer coil unit 700 to the inner coil unit 600.

The main body 200 is implanted in a concave portion formed by excising a portion of the bone tissue 10. The main body 200 converts an electrical signal, received from the inner coil unit 600, into a stimulation signal and transmits the electrical signal to the electrode unit 400.

The electrode unit 400 includes a reference electrode 420 and a stimulation electrode 440.

The electrode unit 400 is connected to the main body 200 to transfer the stimulation signal, generated by the main body 200, to the cochlea, and then the stimulation signal stimulates auditory nerves of the cochlea, so that a user can hear sound due to the stimulated auditory nerves.

One end portion of the reference electrode 420 is connected to the main body 200, and the other end portion of the reference electrode 420 extends to a location adjacent to the main body 200 or the inner coil unit 600 to be implanted in the muscle 20 or the skin 25 while being separated from the stimulation electrode 440 implanted in the cochlea.

One end portion of the stimulation electrode 440 is connected to the main body 200, and the other end portion of the stimulation electrode 440 is inserted into the cochlea while being separated from the reference electrode 420. A plurality of electrode terminals are formed on the surface of the other end portion of the stimulation electrode 440 in the longitudinal direction of the stimulation electrode 440 to be sequentially exposed at predetermined distances from each other. When the other end portion of the stimulation electrode 440 is inserted into the cochlea, the electrode terminals are in contact with auditory nerves within the cochlea.

The inner coil unit 600 is disposed between the muscle 20 and the skin 25 of the head, and serves to receive an electrical signal from the outer coil unit 700 and transfer the electrical signal to the main body 200 through the connector 500.

The extracorporeal device 650 is disposed outside of the skin 25. When ambient sound is received, the extracorporeal device 650 converts the sound into an electrical signal and transmits the electrical signal to the cochlear prosthesis implant package 100.

A location to which the extracorporeal device 650 is attached may vary depending on a location to which the cochlear prosthesis implant package 100 is implanted. In consideration of wirelessly transmitted electrical signals, the extracorporeal device 650 may be attached to a location adjacent to a location in which the cochlear prosthesis implant package 100 is implanted.

The outer coil unit 700 is disposed outside of the skin 25, adjacently to the inner coil unit 600. The outer coil unit 700 wirelessly transmits an electrical signal, generated by the audio processor 800 by converting sound, to the inner coil unit 600.

The audio processor 800 is attached to an outer portion of the ear or the head, in a location adjacent to the outer coil unit 700. The audio processor 800 receives external sound and converts the external sound into an electrical signal. The audio processor 800 may include a microphone to receive external sound and a volume controller to adjust the volume of the received sound.

Accordingly, communications efficiency can be improved by efficient and accurate wireless transmissions of electfical signals between the inner coil unit 600 and the outer coil unit 700 disposed on both sides of the skin 25 to be adjacent to each other. Since an area of the bone tissue 10 required to be excised to accommodate the inner coil unit 600 is reduced, implantation is easy.

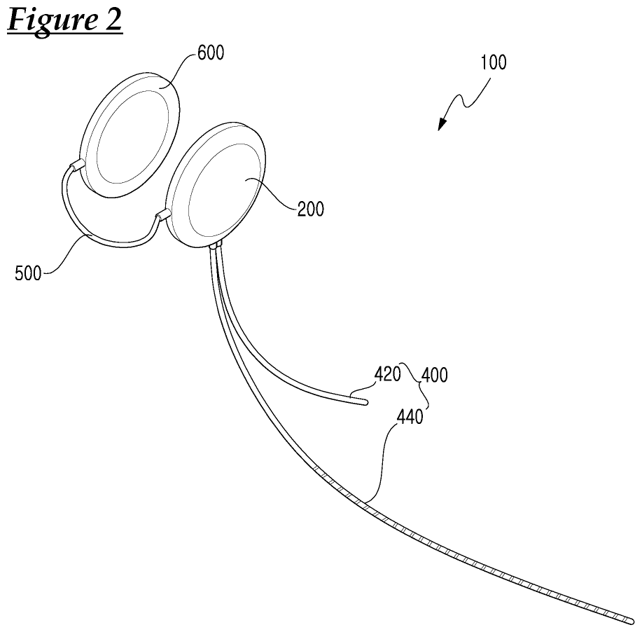

FIG. 2 is a perspective view illustrating the implant package for a cochlear prosthesis illustrated in FIG. 1.

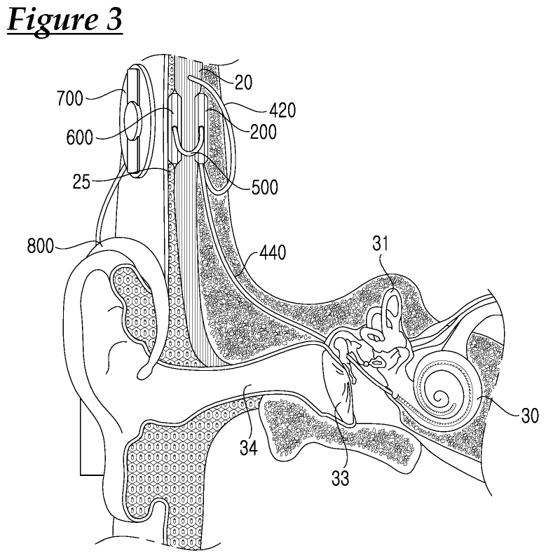

FIG. 3 is a schematic view illustrating the implant package for a cochlear prosthesis illustrated in FIG. 1, implanted in the body of the person.

As illustrated in FIGS. 2 and 3, the main body 200 has the shape of a circular plate, and is inserted into and attached to a portion formed by excising a portion of the bone tissue 10 in a space between the muscle 20 and the bone tissue 10.

The main body 200 serves to receive an electrical signal through the connector 500 and convert the electrical signal to an electrical stimulation signal to stimulate a cochlea 30. The main body 200 is connected to one end of each of the reference electrode 420 and the stimulation electrode 440.

Since the main body 200 has a relatively smaller area compared to a related-art structure in which the inner coil and the main body are connected, it is possible to reduce a procedure time to excise the bone tissue 10, so that an operation of implanting the cochlear prosthesis can be reliably and efficiently carried out.

In addition, the main body 200 may be disposed in a flat location adjacent to the external auditory canal, although the main body 200 may be disposed in a variety of locations, such as a temporal bone, a parietal bone, and a posterior bone of the temporal region.

The reference electrode 420 is made of a wire-shaped material able to transfer an electrical signal, and is disposed to be earthed to the skin 25 or the muscle 20 adjacent to the main body 200. The stimulation electrode 440 is disposed on the surface of the plurality of exposed externally electrode terminals to stimulate auditory nerves in the cochlea 30.

The stimulation electrode 440 is inserted into the cochlea 30 while being spirally bent. The stimulation electrode 440 transfers the stimulation signal, converted by the main body 200, to the auditory nerves, so that the person implanted with the cochlear prosthesis implant package 100 can hear sound.

The stimulation electrode 440 passes by the mastoid or the external auditory canal 34 before being inserted into the cochlea 30. A path along which the stimulation electrode 440 is inserted may vary depending on the physical characteristic of the person or the operation environment.

The connector 500 has the shape of a wire electrically connecting the main body 200 and the inner coil unit 600, and extends through the muscle 20 to be connected to the inner coil unit 600. The connector 500 is flexible and is coated with a corrosion resistant material.

The inner coil unit 600 may be disposed in a variety of locations between the skin 25 and the muscle 20 of the temporal region depending on the length of the connector 500, regardless of the position of the main body 200. Thus, the outer coil unit 700 wirelessly connected to the inner coil unit 600 may be disposed in a variety of locations outside of the temporal region.

Accordingly, a time for implantation can be reduced due to the reduced volume of the main body 200 separated from the inner coil unit 600. Since the inner coil unit 600 can be implanted in a variety of locations of the temporal region, separated from the main body 200, the outer coil unit 700 can be fitted to a variety of locations according to the physical condition of the person.

In addition, since the inner coil unit 600 is disposed between the skin 25 and the muscle 20, the outer coil unit 700 and the inner coil unit 600 are disposed to be relatively adjacent to each other, compared to the related art. Thus, the effect of transferring electrical signals can be advantageously improved.

In addition, the outer coil unit 700 may be disposed in a location adjacent to the inner coil unit 600, and the audio processor 800 may be fitted to a variety of locations connected to the outer coil unit 700 via electrical wires.

Furthermore, the inner coil unit 600 may be disposed in the external auditory canal 34, a location adjacent to the external auditory canal 34, or the mastoid without skin incision or tissue peeling, instead of being disposed between the skin 25 and the muscle 20. In this case, an electrical signal generated by the outer coil unit 700 is transferred to the main body 200 through the connector 500, thereby improving communications efficiency, which can advantageously reduce communications errors and extend a battery available time.

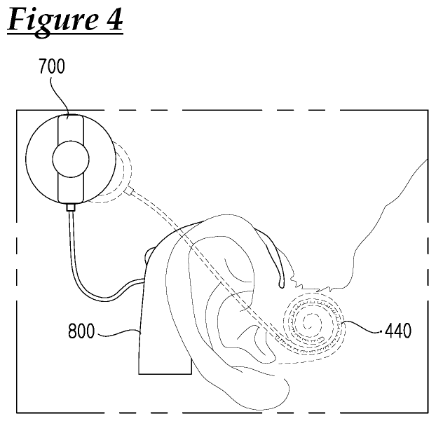

FIG. 4 is a perspective view illustrating a process of implanting the implant package for a cochlear prosthesis illustrated in FIG. 2 in the body of a person.

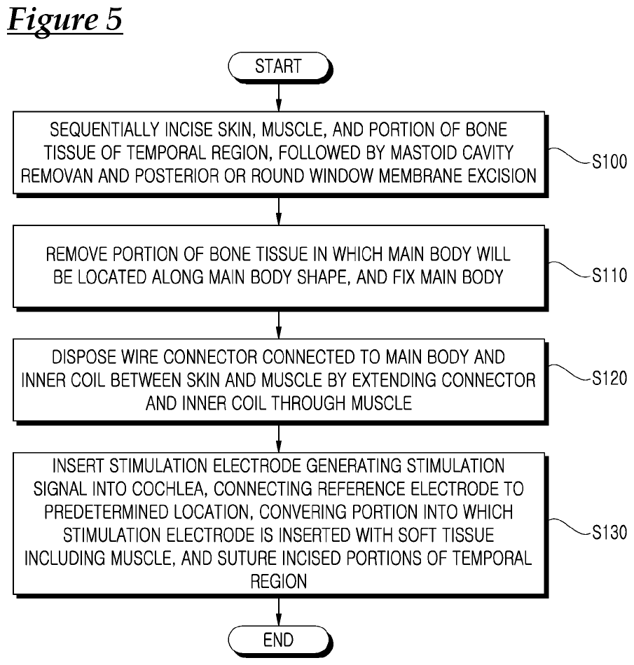

FIG. 5 is a flowchart illustrating a method of implanting the implant package for a cochlear prosthesis illustrated in FIG. 1.

As illustrated in FIGS. 4 and 5, in the method of implanting the cochlear prosthesis implant package 100, in step S100, portions of the skin 25, the muscle 20, and the bone tissue 10 of the temporal region are incised sequentially, followed by mastoid cavity removal and then posterior or round window membrane excision.

In step S110, a portion of the bone tissue 10, in which the main body 200 connected to the reference electrode 420 and the stimulation electrode 440 and converting an electrical sound signal into an electrical stimulation signal will be located, is removed along the shape of the main body 200, and then the main body 200 is fixed to the bone tissue 10.

Since the main body 200 is separated from the inner coil unit 600 unlike the related art, the volume of the main body 200 is relatively small, and thus a time for excising the bone tissue 10 to insert the main body 200 is advantageously reduced.

In step S120, the wire-shaped connector 500 connected to the main body 200 and the inner coil of the inner coil unit 600 are extended through the muscle 20 to be disposed between the skin 25 and the muscle 20.

In step S130, the stimulation electrode 440 for generating stimulation signals is inserted into the cochlea 30, the reference electrode 420 is connected to a location adjacent to the main body 200, the portion into which the stimulation electrode 440 is inserted is covered with a soft tissue of the skin 25 including the muscle 20, and the incised portions of the temporal region are sutured.

In this case, the inner coil unit 600 may be implanted to be disposed in a location in which the thickness of the skin 25 is relatively low. This can consequently improve the transmission of electrical signals, thereby advantageously improving communications efficiency or reducing communications errors.

In the step of implanting the inner coil unit 600 between the skin 25 and the muscle 20, the inner coil unit 600 may be disposed between the skin 25 and the muscle 20, corresponding to a variety of locations of the temporal region, while being separated from the main body 200, depending on the length of the connector 500.

In addition, the inner coil unit 600 may be disposed in the external auditory canal 34, a location adjacent to the external auditory canal 34, or the mastoid without skin incision or tissue peeling, instead of being disposed between the skin 25 and the muscle 20.

Furthermore, the outer coil unit 700 is disposed outside of the head and connected to the audio processor 800, which converts sound into electrical signals, to transmit the electrical signals toward the inside of the head, and is attached to an outer portion of the ear or the head to be adjacent to the inner coil unit 600.

In addition, in step S130 of implanting the inner coil unit 600, the inner coil unit 600 may be disposed in a location of the temporal region, in which the thickness of the skin is relatively low and the bone tissue is flat. In step S120 of implanting the main body 200, the main body 200 may be disposed in a location of the temporal region, in which the thickness of the muscle is relatively low and the bone tissue is flat.

Although the foregoing exemplary embodiments of the present disclosure have been described with reference to the accompanying drawings, the embodiments described in the specification and the configurations illustrated in the drawings are merely best modes of the present disclosure, rather than representing all aspects of the scope of the present disclosure. It should be understood that a variety of equivalents and alternative embodiments could be made at a point in time at which the present application was filed. Accordingly, the foregoing embodiments shall be interpreted as being illustrative, while not being limitative, in all aspects. It should be understood that the scope of the present disclosure shall be defined by the appended Claims rather than by the foregoing embodiments, and that all of modifications and alterations derived from the definition of the Claims and their equivalents fall within the scope of the present disclosure.

It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

What is claimed is:

1. An implant package for a cochlear prosthesis, the implant package being implanted in a head of a person to be adjacent to an extracorporeal device comprising an audio processor converting sound into an electrical signal and an outer coil unit transmitting the electrical signal into the head, the implant package comprising:
an inner coil unit receiving the electrical signal transmitted by the outer coil unit;
a main body connected to the inner coil unit to convert the electrical signal into an electrical stimulation signal; and
an electrode unit connected to the main body and inserted into a cochlea to stimulate auditory nerves of the cochlea using the electrical stimulation signal,
wherein the inner coil unit is separated from the main body to be disposed between skin and muscle of the head.

2. The implant package according to claim 1, wherein the main body is connected to the inner coil unit via a conductive wire, and is separated from the inner coil unit to be implanted in a bone tissue of the head between the muscle and the bone tissue of the head.

3. The implant package according to claim 1, wherein the inner coil unit is disposed in a location in which the bone tissue has a flat surface.

4. The implant package according to claim 3, wherein the inner coil unit is disposed in a location adjacent to or spaced apart from the main body.

5. The implant package according to claim 1, wherein the inner coil unit is disposed in the external auditory canal, a location adjacent to the external auditory canal, or the mastoid without skin incision or tissue peeling.

6. A method of implanting a cochlear prosthesis, the method comprising:
sequentially incising portions of the skin, a muscle, and a bone tissue of a temporal region, followed by mastoid cavity removal and then posterior or round window membrane excision;
removing a portion of the bone tissue, in which a main body connected to a reference electrode and a stimulation electrode to convert an electrical sound signal into an electrical stimulation signal is to be located, along the shape of the main body, and fixing the main body to the bone tissue;
disposing a wire-shaped connector connected to the main body and an inner coil of an inner coil unit between the skin and the muscle by extending the connector and the inner coil unit through the muscle; and
inserting the stimulation electrode generating the stimulation signal into the cochlear, connecting the reference electrode to a predetermined location, covering a portion into which stimulation electrode is inserted with a soft tissue including the muscle, and suturing the incised portions of the temporal region.

7. The method according to claim 6, further comprising attaching an outer coil unit attached to an outer portion of the head of a person to be adjacent to the inner coil unit, the outer coil unit being connected to an audio processor for converting sound into electrical signals to transmit the electrical signals into the head.

8. The method according to claim 6, wherein, in the disposing of the inner coil unit, the inner coil unit is disposed in an external auditory canal, a location adjacent to the external auditory canal, or a mastoid without skin incision or tissue peeling, instead of being disposed between the skin and the muscle.

9. The method according to claim 6, wherein, in the fixing of the main body, the main body is disposed in a location of the head of a person, in which the thickness of the muscle is relatively low and the bone tissue is flat.
